(19) United States
(12) Patent Application Publication
(10) Pub. No.: US 2010/0113943 A1
(43) Pub. Date: May 6, 2010

(54) SYSTEM AND METHOD FOR SIMULTANEOUS CENTRAL AND BRACHIAL ARTERIAL PRESSURE MONITORING

(76) Inventors: John E. Burnes, Coon Rapids, MN (US); Mustafa Karamanoglu, Fridley, MN (US)

Correspondence Address: MEDTRONIC, INC., 710 MEDTRONIC PARKWAY NE, MINNEAPOLIS, MN, 55432-9924, US

(21) Appl. No.: 12/262,349

(22) Filed: Oct. 31, 2008

(51) Int. Cl. A61B 5/0215

(52) U.S. Cl. 600/486

(57) ABSTRACT

An implantable medical device system and corresponding method to monitor blood pressure by transforming a measured pressure signal to estimate a blood pressure metric or waveform corresponding to a target site. An implantable sensor generates a signal corresponding to blood pressure at a first arterial branch location and a processor receiving the signal applies a transfer function to the signal to derive a blood pressure metric or waveform at a target site.

### TECHNICAL FIELD

The disclosure relates generally to implantable medical devices and, in particular, to an implantable medical device for ambulatory monitoring of blood pressure.

### BACKGROUND

Brachial blood pressure measured using an upper arm cuff is the standard blood pressure measurement relied on by clinicians for assessing and managing patients having diseases or conditions affecting blood pressure, such as hypertension. Clinicians tend to have the greatest familiarity with brachial cuff pressure data and its implications in therapy management. Brachial blood pressure measurements, however, are typically performed at isolated times, measured in the clinic or by a patient using a home monitor, and generally provide only a systolic and a diastolic pressure. Central aortic blood pressure provides potentially greater prognostic value than brachial blood pressure. Central blood pressure measurements in the ascending aorta, however, require invasive procedures, which can be costly, time consuming, and pose inherent risks to the patient.

### BRIEF DESCRIPTION OF THE DRAWINGS

FIG. 1 is a schematic diagram of an implantable medical device (IMD) coupled to a first lead carrying a pressure sensor and a second lead carrying a second pressure sensor.

FIG. 2 is a functional block diagram of one embodiment of the IMD shown in FIG. 1.

FIG. 3 is a flow chart of a method for monitoring blood pressure for use in an implantable medical device.

FIG. 4 is a flow chart of another method for monitoring blood pressure for use in an implantable medical device.

### DETAILED DESCRIPTION

In the following description, references are made to illustrative embodiments. It is understood that other embodiments may be utilized without departing from the scope of the invention. For purposes of clarity, the same reference numbers are used in the drawings to identify similar elements. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

As used herein, the term “pressure metric” refers to any feature or index that can be determined from a blood pressure waveform such as diastolic, systolic, maximum peak, minimum peak, mean pressure, systolic time index, diastolic time index or the like. The blood pressure waveform from which a pressure metric is determined may be an actual measured pressure waveform or an estimated pressure waveform.

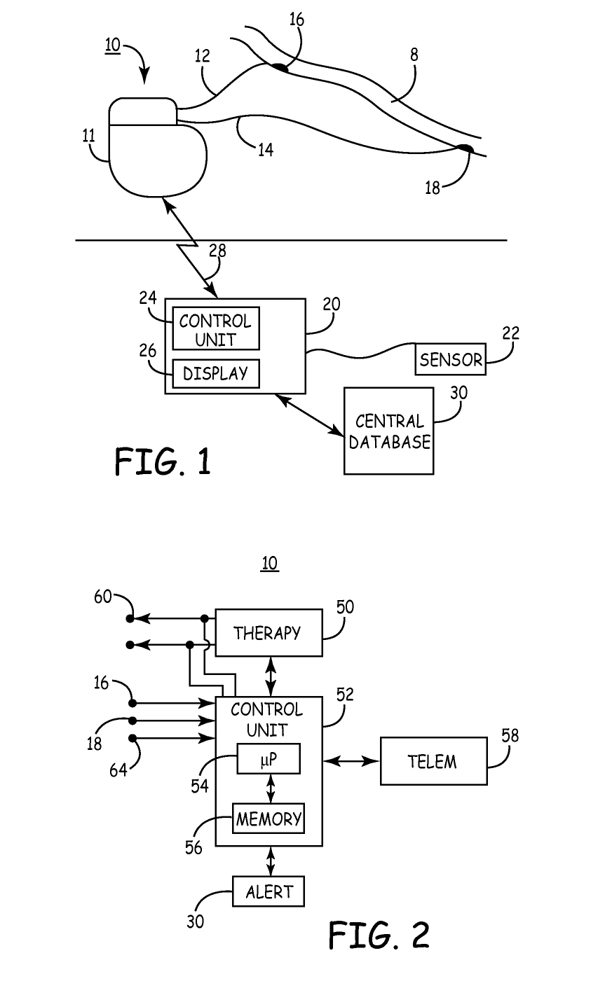

FIG. 1 is a schematic diagram of an implantable medical device (IMD) 10 for ambulatory monitoring of blood pressure. A single pressure signal may be sensed and used to provide both central and peripheral artery pressure data. As will be described herein, blood pressure waveforms, waveform features and/or pressure waveform metrics relating to a targeted pressure monitoring site is estimated using ambulatory pressure monitoring at a measurement site remote from the targeted site. A targeted pressure monitoring site is a site of interest to a clinician for gathering pressure data. The measurement site is different from the targeted site and may be either more central or more peripheral from the target site.

In one embodiment, it is desired to provide central aortic pressure data and brachial artery pressure data. Central aortic pressure data provides potentially more valuable prognostic and diagnostic information than brachial artery pressure, but clinicians tend to have greater familiarity with brachial pressure data. Accordingly, in one embodiment, one target site is the ascending aorta and another target site is the brachial artery and a measurement site is located at any surgically accessible arterial site between the two target sites. The ambulatory pressure monitoring provides frequent and even continuous or semi-continuous central blood pressure data, which can be viewed and analyzed in relation to simultaneously generated brachial pressure data. Determining brachial blood pressure data along with the central blood pressure data provides clinicians with the data they are familiar along with the potentially more valuable central blood pressure data.

IMD 10 is coupled to a first lead 12 carrying a pressure sensor 16. IMD 10 may be coupled to a second lead 14 carrying a second pressure sensor 18. Pressure sensor 16 is positioned at a measurement site for acquiring a signal from which a target site pressure waveform or waveform features are estimated. Pressure sensor 16 is a wholly implantable sensor used for ambulatory monitoring of the measurement site pressure signal as opposed to transcutaneous catheters or other partially implanted devices implanted acutely for monitoring pressure at discrete time intervals and which require the patient to be fully or partially immobilized. Pressure sensor 16 is coupled directly to IMD 10, either by a lead or by telemetric communication. Pressure sensor 18, when used, is positioned at a reference site for acquiring a pressure signal that can be used to calibrate signals measured by sensor 16 and/or for updating transfer functions used to compute estimated target site pressure data.

Pressure sensors 16 and 18 are shown positioned at spaced apart locations along an arterial branch 8. Pressure sensors 16 and 18 are embodied as any sensor capable of generating a signal correlated to blood pressure at the respective arterial location.

Pressure sensors 16 and 18 may be embodied as sensors positioned along or adjacent the exterior surface of the artery 8. Such sensors may include an acoustical sensor, a displacement sensor, an electromagnetic sensor, a strain gauge, an ultrasound transducer or any other sensor generating a signal correlated to blood pressure at the arterial site. Alternatively, one or both of pressure sensors 16 and 18 may be embodied as indwelling sensors positioned within the lumen of artery 8. Such sensors may include a MEMS device, a strain gauge, capacitive sensor or the like. A pressure sensor suitable for chronic implantation in a blood volume is described in U.S. Pat. No. 5,564,434 (Halperin), incorporated herein by reference in its entirety. It is recognized that sensors 16 and 18 may incorporate various coatings or drug releasing mechanisms for preventing infection, thrombosis, and excessive encapsulation of the sensor.

In FIG. 1, sensors 16 and 18 are shown as lead-based sensors, each carried by a separate lead 12 and 14. Leads 12 and 14 carry conductors extending from IMD 10 to sensors 16 and 18 for carrying signals from sensors 16 and 18 to circuitry (not shown) enclosed within IMD housing 11. Leads 12 and 14 may be tunneled to deploy sensors 16 and 18 at desired locations along the exterior surface of artery 8. Alternatively, leads 12 and/or 14 may be advanced along a lumen of artery 8 or through the wall of artery 8 such that sensor 16 and/or 18 is positioned within the lumen of artery 8.

Sensors 16 and 18 may be carried by a single lead instead of two separate leads as shown in FIG. 1. Sensors 16 and 18 could be spaced apart at two locations along a single lead body such that when the lead body is tunneled along the exterior wall or within the lumen of an artery the sensors 16 and 18 are positioned at spaced apart locations along the artery. Sensors 16 and 18 may alternatively be located along two separate branches of a bifurcated lead body for positioning at two spaced apart locations along artery 8. In still other embodiments, sensors 16 and 18 may be leadless sensors capable of transmitting signals wirelessly to IMD 10 and/or to an external device 20.

Sensors 16 and 18 may be positioned along the aorta (ascending or descending), a subclavian artery, mammary artery, carotid artery, axillary artery, radial artery, brachial artery or any other arterial location accessible for operative positioning of the pressure sensors 16 and 18. While sensors 16 and 18 are schematically shown spaced apart along a single arterial branch 8, sensors 16 and 18 may be positioned along different arterial branches as long as one sensor is positioned relatively more centrally in the arterial system than the other sensor. In other words, one sensor is further “downstream” in the arterial system from the ascending aorta than the other sensor. For example, one sensor may be positioned along the mammary artery and another along the radial artery. In another example, one sensor 16 may be positioned along the aorta (ascending or descending) and the other sensor 18 along a peripheral arterial branch.

As will be described herein, one sensor 16 provides a measurement signal used for deriving blood pressure metrics and/or blood pressure waveforms at a target site using a transfer function. The second sensor 18 when present is used to provide a reference signal to update the transfer function and optionally calibrate the measurement signal. The second sensor 18 may be implemented as a MEMs device that is activated relatively infrequently only to acquire signal data used for updating a transfer function.

The measurement sensor 16 can be more peripheral or more central than the reference sensor 18 as long as the two sensors are spaced far enough apart to allow a transfer function and changes in the transfer function or transit times between the two locations to be determined. This spacing between sensors may be a few centimeters or more. The reference and measurement sensors are generally positioned along a common arterial tree, such as along the upper chest or shoulder, in contrast to two distinct arterial trees, such as one in the upper chest and one in the lower leg.

IMD 10 is capable of bidirectional wireless telemetry with an external device 20. External device 20 may be embodied as a home monitor, a programmer, an external blood pressure monitor or other device for receiving data from IMD 10. Blood pressure data accumulated by IMD 10 may be transmitted to external device 20 and made available to a clinician for display and further analysis. External device 20 includes a control unit 24, which may include a microprocessor and memory, for processing data received from IMD 10, and controlling display of data on optional display 26.

External device 20 may further include or be coupled to an external pressure sensor 22 used for acquiring external pressure data. External pressure sensor 22 may be embodied as an external cuff sphygmomanometer, ultrasound transducer, a tonometer or a transcutaneous arterial cannula. External pressure sensor 22 may be used for measuring blood pressure at a brachial artery location or a fingertip. External pressure data may be transmitted to IMD 10 by external device 20 and used for adjusting a transfer function and/or calibrating measured pressure signals or derived pressure data. External pressure data may alternatively be used by control unit 24 or another external processor in combination with data received from IMD 10 for calibrating and processing internal pressure data acquired by IMD 10.

External pressure sensor 22 may additionally or alternatively include an atmospheric pressure sensor for storing ambient pressure data with time and date stamps or for transmitting in real time ambient pressure data to IMD 10. Ambient pressure data may be used to adjust absolute pressure signals and derived waveforms or blood pressure metrics to relative pressure. Reference is made, for example, to the above-referenced U.S. Pat. No. 5,564,434 (Halperin) and to U.S. Pat. No. 7,376,951 (Bennett), incorporated herein by reference in its entireties.

External device 20 is shown in communication with a central database 30 via communication link 32, which may be a wireless or hardwired link. Programming data to be transferred to IMD 10 and interrogation data received from IMD 10 may be transmitted between external device 20 and central data base 30 via link 32. Central database 30 may be a centralized computer or a web-based or other networked database used by a clinician for remote patient monitoring and management. Various methods described herein for deriving a blood pressure signal or blood pressure metrics at a target site may be implemented in one or more of the IMD system components shown in FIG. 1, namely in the IMD 10, external device 20 and/or central database 30, and may include any combination of hardware, firmware and/or software.

FIG. 2 is a functional block diagram of one embodiment of IMD 10 shown in FIG. 1. IMD 10 generally includes a control unit 52 which may employ microprocessor 54 and associated memory 56 for controlling device functions. IMD 10 may include a therapy delivery module 50 for delivering a therapy in response to determining a need for therapy, e.g., based on sensed physiological signals. Therapy delivery module 50 may provide drug delivery therapies or electrical stimulation therapies, such as cardiac pacing or anti-arrhythmia therapies. As such, therapy module 50 is coupled to one or more therapy deliver devices, for example embodied as electrodes 60. Electrodes 60 may be carried by IMD housing 11 (shown in FIG. 1) or by a lead extending from IMD 10. Therapy delivery devices coupled to module 50 may alternatively or additionally include a drug delivery port, for example provided by a catheter extending from IMD 10. Therapies are delivered by module 50 under the control of control unit 52.

Control unit 52 may also receive signals from electrodes 60, for example cardiac electrogram signals may be sensed by electrodes. Cardiac electrical signals may be monitored for use in diagnosing or managing a patient condition or may be used for determining when a therapy is needed and controlling the timing and delivery of the therapy. Control unit 52 may include sense amplifiers and other signal processing circuitry such as an analog-to-digital converter for processing sensed signals. Electrical signals may be used by microprocessor 54 for determining a heart rate and detecting physiological events, such as detecting and discriminating cardiac arrhythmias.

Control unit 52 may additionally receive signals from other physiological sensors 64, such as an activity sensor, posture sensor, pH sensor, temperature sensor, blood chemistry sensor, or the like. Such secondary sensors may be used to monitor secondary physiological conditions which vary with or influence blood pressure.

IMD 10 is additionally coupled to one or more blood pressure sensors 16 and 18 for monitoring a patient's blood pressure. In particular, microprocessor 54 applies a transfer function stored in memory 56 to a signal sensed by measurement sensor 16 to transform the measured pressure waveform to a pressure waveform at a target site. While the methods described herein refer primarily to applying a transfer function, it is recognized that this transformation may be performed using either a transfer function in the frequency domain or a convolution in the time domain.

A reference transfer function is initially stored in memory 56 relating the measurement site to the reference site. The stored reference transfer function may be based on a mathematical model or derived through analysis of actually recorded measurement site and reference site waveforms. Additional transfer functions are stored in memory 56 relating the measurement site to each of the target sites. A stored target site transfer function, i.e. a transfer function relating the measurement site to a target site, may be updated using a measurement site signal and a reference signal sensed by reference sensor 18. By simultaneously measuring a pressure waveform at the measurement site and at the reference site, and applying the stored reference transfer function, a determination can be made if the stored transfer function should be updated. The reference transfer function may be applied to transform the measurement site waveform to an estimated reference site waveform. The estimated reference site waveform is then compared to the actual reference site waveform. Alternatively, the reference transfer function may be applied to transform the reference site waveform to an estimated measurement site waveform.

The estimated and actual waveforms are then compared. If the estimated and actual waveforms match, the stored transfer functions are considered to be correct. If the estimated and actual waveforms do not match, the stored reference transfer function is adjusted until the estimated and actual waveforms do match. The adjustments made to the stored reference transfer function can then be applied to the stored target site transfer functions. Adjustments to stored transfer functions may include adjusting wave propagation and reflection coefficients.

The updated transfer function data may be transferred to an external device using telemetry unit 58. The propagation properties of the blood pressure wave may change over time due to disease progression, drug effects or other factors. For example, arterial wall stiffness may change with disease. Such changes will be reflected in the transfer function. Thus the trends in transfer function coefficients can be useful in diagnosing and managing patient disease.

A blood pressure monitoring algorithm may be stored in memory 56 and executed by microprocessor 54 with input received from electrodes 60 and pressure sensors 16 and 18. Pressure sensing may occur in a timed relationship to an ECG/EGM signal received from electrodes 60. In one embodiment, microprocessor 54 is configured to execute software-implemented filtering operations for deriving a blood pressure metric or waveform corresponding to one or more target sites. Alternatively, analog or digital filters, such as finite impulse response (FIR) filters may be implemented for applying a transfer function to a sensed blood pressure signal.

In another embodiment, a look up table (LUT) may be stored in memory 56 and accessed by microprocessor 54. LUT values may be derived for an individual patient based on physiological measurements and anatomic measurement, including, for example, calibrated blood pressure measurements made at two or more sites. LUT values may additionally or alternatively be selected from a general LUT developed for a patient population.

Blood pressure data may be stored for use in diagnosing or monitoring the patient or for determining the need for delivering a therapy. Memory 56 is utilized for storing a variety of programmed-in operating modes and parameter values that are used by control unit 52. The memory 56 may also be used for storing data compiled from sensed physiological signals and/or relating to device operating history for telemetry out on receipt of a retrieval or interrogation instruction. Control unit 52 may respond to the blood pressure data by altering a therapy, triggering data storage, enabling other sensors for acquiring physiological data, or triggering alert 62 to generate an alert signal or notification to the patient or a caregiver that a serious condition has been detected, which may require medical intervention. Data relating to sensed blood pressure signals or derived blood pressure waveforms or metrics may be stored in memory 56 for later retrieval.

IMD 10 further includes telemetry unit 58. Programming commands or data are transmitted during uplink or downlink telemetry between IMD telemetry unit 58 and external telemetry circuitry included in external device 20 shown in FIG. 1.

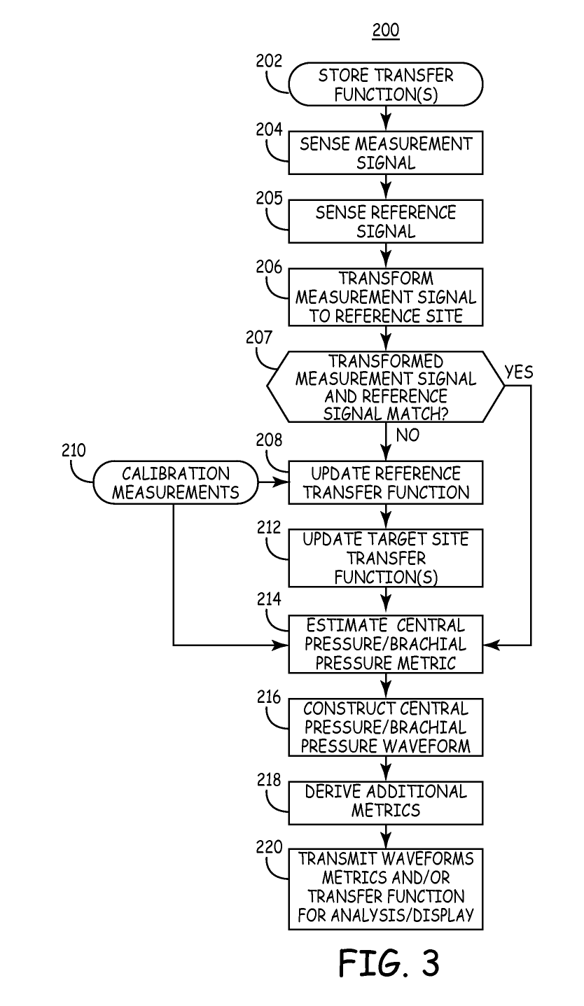

FIG. 3 is a flow chart of a method for monitoring blood pressure in an implantable medical device. Flow chart 200 is intended to illustrate the functional operation of the device, and should not be construed as reflective of a specific form of software or hardware necessary to practice the embodiments described herein. It is believed that the particular form of software will be determined primarily by the particular system architecture employed in the device and by the particular detection and therapy delivery methodologies employed by the device. Providing software to accomplish the functionality described, in the context of any modern implantable device given the disclosure herein, is within the abilities of one of skill in the art.

Methods described in conjunction with flow charts presented herein may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 202, one or more target site transfer functions are stored in IMD memory. Each stored target site transfer function relates a blood pressure metric and/or waveform at a target site, central or peripheral, to a blood pressure signal at a measurement site. As described above, the transfer functions may be stored in the form of a look-up table. In one embodiment, a transfer function relating the blood pressure signal at a measurement site to central ascending aortic pressure and a transfer function relating the measurement site blood pressure signal to brachial artery pressure are stored. The measurement site may be intermediate the ascending aorta and brachial artery, for example, the radial, mammary, subclavian or carotid artery.

In addition, a reference transfer function may be stored at block 202 relating a blood pressure metric or waveform at the measurement site to the analogous blood pressure metric or waveform a reference site. The reference transfer function may be updated on a periodic basis and used to update the stored target transfer functions. As described above, the reference site is the location of a reference pressure sensor. When the reference pressure sensor is implanted, it may be an extravascular or indwelling sensor positioned along the same arterial branch, but spaced apart from, the measurement site. An implanted reference pressure sensor may alternatively be located along a different arterial branch than the measurement sensor but along the same arterial tree. A reference pressure sensor may alternatively be an external sensor, such as a brachial cuff or fingertip pressure sensor. A reference pressure sensor may be an internal or external pressure sensor positioned to measure a pressure signal at one of the target sites. The reference site is therefore not required to be different than the target sites, but is different than the measurement site.

At block 204, a measurement blood pressure signal is sensed using a pressure sensor implanted at the arterial measurement site. As discussed above, the measurement site may be along the aorta, a subclavian, mammary, carotid, axillary, radial, brachial or any other arterial location accessible for operative positioning of the implanted, indwelling or extravascular pressure sensor.

The measurement site blood pressure may be measured on a continuous, periodic, or triggered basis. Continuous monitoring allows continuous central and brachial artery (or other target site) pressure metrics or waveforms to be estimated. Alternatively time-gated monitoring may be performed to acquire the measurement signal at one or more particular times in the cardiac cycle. Sensing windows may be set based on timing of sensed ECG/EGM events.

At block 205, a reference blood pressure signal is sensed using the reference pressure sensor. The reference blood pressure signal may be determined only on a periodic basis to allow periodic updates of stored transfer functions and/or calibration of the measurement site signal or the estimated target site pressure waveform or metrics when the reference pressure sensor is a calibrated sensor.

At block 206, the measurement signal is transformed using the stored reference transfer function to estimate a blood pressure metric or pressure waveform at the reference site. At block 207, the transformed signal is compared to the actual reference signal sensed at block 205. If the transformed signal matches the actual reference signal within a predetermined percentage or acceptable margin of error, method 200 advances to block 214 to estimate the target site pressure waveform or metrics. Comparisons made to determine a match between the transformed signal and an actual measured signal may include comparisons between the waveform morphologies, waveform peak magnitudes, peak widths, slopes, mean values or other waveform characteristics.

If the transformed signal does not match the actual reference signal, the stored reference transfer function is updated at block 208. The stored transfer function may be iteratively adjusted until the transformed measurement signal matches the reference signal. An updated reference transfer function may be computed as the relationship between one or more features of the measured blood pressure waveform and the corresponding feature(s) of the reference blood pressure waveform, for example peak systolic pressure, diastolic pressure, or mean pressure. Methods for computing transfer functions relating to arterial pressure waveforms are generally described in Karamanoglu, M. et al., “On-line Synthesis of the Human Ascending Aortic Pressure Pulse From the Finger Pulse”, Hypertension 1997;30:1416-1424, hereby incorporated herein by reference in its entirety.

Alternatively, the reference transfer function is computed to relate the sampled reference waveform to the sampled measurement waveform. Fourier transform analysis or other methods may be used for deriving the transfer function in the frequency domain. In other embodiments, a mathematical model of the blood flow mechanics, such as an equivalent element model, is used compute a transfer function relating the measurement site waveform or metric(s) to the reference site waveform or metric(s).

Updating the reference transfer function may take into account calibration measurements as indicated at block 210. Calibration measurements may be performed at the time of implantation and may be repeated periodically using an external device or an indwelling catheter when the reference sensor itself is not a calibrated sensor.

The calibration measurements at block 210 may further include adjusting the pressure measurements for ambient pressure by subtracting a measured or estimated atmospheric pressure from the measured blood pressure. Calibration may be performed as an iterative procedure between the IMD measuring pressure at a measurement site, a brachial cuff sphygmomanometer or other external, calibrated pressure measurement, and an external device communicating between the IMD and external pressure measurement device. A peak, trough, mean or other feature of the pressure waveform recorded by the measurement sensor can be adjusted according to analogous values measured by the calibrated pressure measuring device. Alternatively, the entire calibrated waveform recorded by a calibrated instrument can be used to calibrate the waveform measured by the measurement sensor 18.

At block 212, the stored target site transfer functions are updated based on the updated reference transfer function. Changes in coefficients of a reference transfer function can be used to update coefficients in the target site transfer functions relating the measurement site to the target sites. In particular, an updated reference transfer function is used adjust the stored target site transfer functions by adjusting coefficients relating to the wave propagation speed and the amount of wave reflection.

It is recognized that blocks 205 through 212 will typically be performed only on a periodic basis and not every time target site pressure data is to be accumulated. Pathological, pharmacological and aging effects may alter the waveform propagation and reflection properties of the arterial system over time such that the transfer function relating the measurement site to the target sites becomes altered. However, these changes are expected to be gradual and, as such, transfer function updates may only need to be performed on a relatively infrequent basis such as weekly or monthly. When transfer function updates are not performed, method 200 may advance directly from block 205 to block 214.

It is further recognized that the target site transfer function updates are optional. As such, blocks 205 through 212 may be omitted. A sensed measurement signal (block 205) is transformed at block 214 using stored target site transfer functions to estimate a target site pressure metric or waveform. Uncalibrated measurement site pressure signals may be used for detecting relative changes in estimated pressure metrics or waveforms. In this embodiment, only a measurement sensor implanted for ambulatory monitoring of a measurement site pressure signal is used to estimate target site pressure metrics.

In another embodiment, the measurement sensor signal is used to estimate target site pressure metrics and an external sensor is used to calibrate and/or adjust target site transfer functions. In yet another embodiment, the measurement sensor signal is used to estimate target site pressure metrics and another implanted sensor is used for calibrating the measurement signal or the estimated pressure metrics and/or updating the target site transfer functions. The second implanted sensor may be temporarily implanted for acute reference site pressure monitoring or chronically implanted for ambulatory reference site pressure monitoring.

In still another embodiment, an implanted measurement site sensor is used in combination with an implanted reference site sensor and an external calibration sensor for estimating target site pressure metrics, updating target site transfer functions, and calibrating estimated target site pressure metrics, either before or after transformation of the measurement signal.

At block 214, pressure metrics corresponding to central aortic pressure and brachial artery pressure (or other target sites) are computed using the measurement signal and the originally-stored or the updated target site transfer functions. One or more pressure metrics for each target site may be computed at block 214. Analogous pressure metrics for each target site are computed using the measurement signal to derive pressure metrics that are contemporaneous across each target site. For example, an aortic systolic pressure and a brachial artery systolic pressure are derived using the same measurement signal such that the two estimated systolic pressures correspond to the same point in time.

A pressure metric may be computed using a calibrated measurement signal adjusted for atmospheric pressure or the pressure metric may be calibrated and adjusted for atmospheric pressure after computing the metric from an uncalibrated measurement signal waveform and the transfer function. Calibration measurements at block 210 may then be used to calibrate the estimated pressure metrics.

The estimated pressure metrics for central aortic pressure and brachial artery pressure are determined by applying a transfer function to the corresponding pressure metric determined from the measurement signal. Alternatively, a target site pressure waveform may be estimated by transforming the measurement signal to the target site using the appropriate stored or updated transfer function.

If a waveform is not estimated at block 214 by applying a stored or updated target site transfer function to the measurement site signal, a target site waveform may be constructed at block 216 from the estimated metrics determined at block 214. Additional waveform features are computed based on one or more computed metrics to construct an estimated central aortic pressure waveform and a derived brachial artery pressure waveform.

At block 218 additional blood pressure metrics may be computed from the one or more pressure metrics computed at block 214. For example, a mean and diastolic pressure computed by applying the transfer functions to the measurement signal may be used to compute other pressure metrics, such as systolic pressure, at the central aortic or brachial artery site. Additional metrics computed from derived metrics and/or a constructed waveform may include estimates of peripheral resistance, reflection indices, estimates of flow, and the amount of pressure wave augmentation. Metrics that cannot be directly estimated using the pressure metrics estimated at block 214, such as the tension time index and diastolic time index, can be determined from an estimated waveform constructed at block 216. The derived metrics or trends of the derived metrics may be stored along with measured and derived blood pressure waveforms.

At block 218, the IMD may also automatically determine when the estimated pressure at a target site is increasing or decreasing concurrently with pressure at the measurement site or when an estimated target site pressure is changing in a different direction or at a different rate than at the measurement site or another target site.

At block 220, recorded measurement waveforms, recorded reference waveforms, estimated target site waveforms, estimated target site blood pressure metrics, and updated reference transfer function data may be transmitted to an external device for display and/or further analysis by a clinician. Transfer function data relating to the reference transfer function and adjusted target site transfer functions may include useful information for a clinician for tracking changes in arterial physiology. Estimated waveforms, blood pressure metrics, and/or target site transfer function data may then be used in managing patient medications, diet, or other care and/or in adjusting therapies delivered by the IMD.

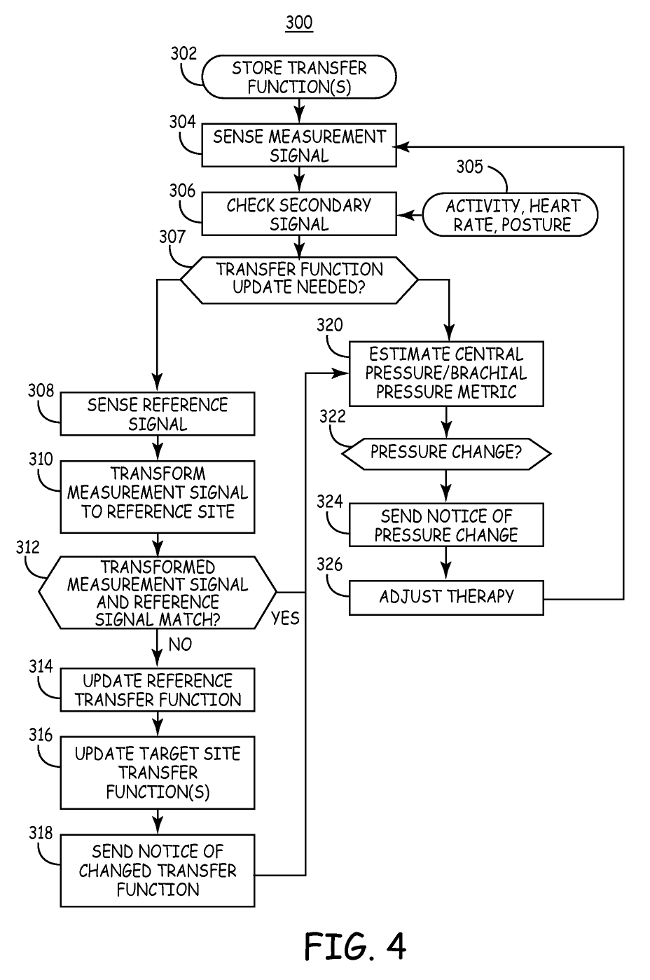

FIG. 4 is a flow chart of an alternative method for simultaneous monitoring of central aortic pressure and brachial pressure for use in an implantable medical device. As generally described in conjunction with FIG. 3, target site transfer functions are stored at block 302. The target site transfer functions relate a blood pressure metric and/or waveform at each of the target sites, namely central aortic and brachial in this example, to a blood pressure signal at a selected measurement site. In addition, a reference transfer function may be stored at block 302 relating a blood pressure metric or waveform at the measurement site to the analogous blood pressure metric or waveform at a reference site. The reference transfer function may be updated on a periodic basis and used to update the stored target transfer functions as described in conjunction with FIG. 3.

At block 304, a measurement blood pressure signal is sensed using a pressure sensor implanted at the selected arterial measurement site. At block 306, secondary physiological signals that can influence blood pressure changes are checked. For example, activity, heart rate, and posture signals may be monitored at block 305 to allow a determination of the heart rate, activity level, and posture of the patient at the time the measurement signal is obtained at block 304.

At block 307, a determination is made whether a stored transfer function needs updating. A transfer function update may be made in response to an external command received by the IMD, after a predetermined interval of time, or in response to a change in any of the monitored secondary signals. For example, a reference transfer function may be stored for a patient assuming an upright, resting condition. If the patient is exercising, as determined by the activity level, a new transfer function may be determined or the existing transfer function may be updated at blocks 308 through 316 and stored for the corresponding high activity level. Reference transfer functions may be determined for high and low activity levels, high and low heart rates, and/or upright and non-upright positions. As such, multiple conditional reference transfer functions may be stored relating the blood pressure waveform at the reference site to the measurement site during defined secondary physiological conditions.

The methods performed at blocks 308 through 316 generally correspond to blocks 205 through 212 described above in conjunction with FIG. 3. At block 314, updating a reference transfer function, however, may include storing separate conditional reference transfer functions for different levels or ranges of secondary physiological signals or conditions. At block 316, target site transfer functions are updated. Similar to the reference transfer function, target site transfer function updates may include storing separate conditional transfer functions for different levels of secondary conditions, such as high and low activity, upright and non-upright posture, and high and low heart rate.

At block 318, any change to stored transfer functions may trigger a notification sent by the IMD to an external device or remote patient management database to alert the patient or clinician of a potential change in the patient's cardiovascular condition.

When transfer function updates are not needed, method 300 may advance directly to block 320 to estimate central and brachial pressure metrics by applying stored target site transfer functions to the measurement signal. The transfer functions applied may be conditional transfer functions selected to correspond to the secondary signal checked at block 306. For example, if the current activity level is high, a target site transfer function stored for high activity is applied to estimate the target site blood pressure metric.

At block 322, a determination is made if a clinically significant blood pressure change is detected. If either of the target site estimated blood pressure metrics exceeds a normal physiological level or some predetermined threshold, a notification may be generated at block 324 and/or a therapy may be adjusted at block 326. A blood pressure change detected at block 322 may also include detecting changes in the ratio or difference between the estimated central aortic pressure and the brachial pressure metrics. If a ratio or difference between the estimated target metrics is exhibiting an increasing or decreasing trend, a notification or therapy adjustment response may be provided. A notification allows a clinician to recognize when the central aortic pressure, which may have greater prognostic and diagnostic value than brachial pressure, may be changing in a different manner than brachial pressure.

Thus, an implantable medical device system and associated methods for ambulatory blood pressure monitoring have been presented in the foregoing description with reference to specific embodiments. It is appreciated that various modifications to the referenced embodiments may be made without departing from the scope of the invention as set forth in the following claims.

We claim:

1. An implantable medical device system, comprising:
a first wholly implantable sensor generating a blood pressure signal at a first arterial location;
a memory storing a first transfer function relating the blood pressure signal to a first target site at a second arterial location spaced apart from the first arterial location;
a processor receiving the blood pressure signal and applying the first transfer function to the blood pressure signal to derive a blood pressure metric corresponding to the first target site; and
a telemetry unit for transmitting data corresponding to the blood pressure metric to an external device.

2. The system of claim 1, further comprising a second sensor generating a reference blood pressure signal at a reference site spaced apart from the first arterial location and the first target site, wherein the processor adjusts the first transfer function in response to the reference signal.

3. The system of claim 2, wherein the second sensor is an external sensor, the system further comprising means for receiving an externally measured reference signal from the second sensor.

4. The system of claim 2, wherein the processor adjusts the blood pressure signal for atmospheric pressure using the externally measured reference signal.

5. The system of claim 2, wherein the second sensor is an implantable sensor.

6. The system of claim 2, wherein the memory stores a reference transfer function relating the blood pressure signal to the reference site, and wherein the processor transforms the blood pressure signal to an estimated signal at the reference site, compares the estimated signal to the reference signal, updates the reference transfer function in response to the comparison, and updates the first transfer function in response to the updated reference transfer function.

7. The system of claim 6, wherein the stored first transfer function comprises a wave propagation coefficient and a reflection coefficient, and
the processor updates the first transfer function by updating one of the wave propagation coefficient and the reflection coefficient.

8. The system of claim 6, further comprising a third sensor generating a signal corresponding to a blood pressure-related physiological condition,
wherein the processor receives the third sensor signal and updates the reference transfer function to include a conditional transfer function in response to the third sensor signal.

9. The system of claim 1, wherein the processor constructs an estimated blood pressure waveform corresponding to the first target site using the derived blood pressure metric.

10. The system of claim 9, wherein the processor computes a new blood pressure metric different than the derived blood pressure metric from the estimated blood pressure waveform.

11. The system of claim 1, wherein the memory stores a second transfer function relating the blood pressure signal to a second target site at a third arterial location spaced apart from the first and the second arterial locations, and wherein the processor applies the second transfer function to the blood pressure signal to derive a blood pressure metric corresponding to the second target site.

12. The system of claim 11, wherein the first target site is an aortic site and the second target site is a brachial artery site, and wherein the processor derives contemporaneous blood pressure metrics for the first target site and the second target site.

13. A method for use in an implantable medical device system, comprising:
sensing a blood pressure signal using a wholly implantable sensor at a first arterial location;
storing a first transfer function relating the blood pressure signal to a first target site at a second arterial location spaced apart from the first arterial location;
applying the first transfer function to the blood pressure signal to derive a blood pressure metric corresponding to the first target site; and
transmitting data corresponding to the blood pressure metric to an external device.

14. The method of claim 13, further comprising:
sensing a reference blood pressure signal at a reference site spaced apart from the first arterial location and the first target site; and
adjusting the first transfer function in response to the reference signal.

15. The method of claim 14, further comprising:
sensing the reference blood pressure signal externally; and
receiving the externally sensed reference signal from an external sensor.

16. The method of claim 14, further comprising adjusting the blood pressure signal for atmospheric pressure using the externally sensed reference signal.

17. The method of claim 14, further comprising sensing the reference blood pressure signal internally.

18. The method of claim 14, further comprising:
storing a reference transfer function relating the blood pressure signal to the reference site;
transforming the blood pressure signal to an estimated signal at the reference site;
comparing the estimated signal to the sensed reference signal;
updating the reference transfer function in response to the comparison; and
updating the first transfer function in response to the updated reference transfer function.

19. The method of claim 18, wherein storing the first transfer function comprises storing a wave propagation coefficient and a reflection coefficient, and wherein updating the first transfer function comprises updating one of the wave propagation coefficient and the reflection coefficient.

20. The method of claim 18, further comprising:
sensing a signal corresponding to a blood pressure-related physiological condition; and
updating the reference transfer function to include a conditional transfer function in response to the third sensor signal.

21. The method of claim 14, further comprising constructing an estimated blood pressure waveform corresponding to the first target site using the derived blood pressure metric.

22. The method of claim 20, further comprising computing a new blood pressure metric different than the derived blood pressure metric from the estimated blood pressure waveform.

23. The method of claim 13, further comprising:
storing a second transfer function relating the blood pressure signal to a second target site at a third arterial location spaced apart from the first and the second arterial locations; and
applying the second transfer function to the blood pressure signal for deriving a blood pressure metric corresponding to the second target site.

24. The method of claim 22, wherein the first target site is an aortic site and the second target site is a brachial artery site, and further comprising deriving contemporaneous blood pressure metrics for the first target site and the second target site.

25. A computer readable medium for storing a set of instructions which when implemented in an implantable medical device cause the device to:
sense a signal corresponding to a blood pressure using a wholly implantable sensor at a first arterial location;
store a first transfer function relating the blood pressure signal to a first target site at a second arterial location spaced apart from the first arterial location;
apply the first transfer function to the blood pressure signal to derive a blood pressure metric corresponding to the first target site; and
transmit data corresponding to the blood pressure metric to an external device.
